(19) United States
(12) Patent Application Publication
(10) Pub. No.: US 2007/0150045 A1
(43) Pub. Date: Jun. 28, 2007

(54) Methods And Apparatus For Treating Aneurysms And Other Vascular Defects

(76) Inventor: David A. Ferrera, Manhattan Beach, CA (US)

Correspondence Address: INSKEEP INTELLECTUAL PROPERTY GROUP, INC, 2281 W. 190TH STREET, SUITE 200, TORRANCE, CA, 90504, US

(21) Appl. No.: 11/678,544

(22) Filed: Feb. 23, 2007

Related U.S. Application Data
(63) Continuation of application No. 10/400,138, filed on Mar. 25, 2003, now abandoned.

(51) Int. Cl. A61F 2/06

(52) U.S. Cl. 623/1.11; 623/1.15

(57) ABSTRACT

Devices and methods are disclosed for placing a barrier across the neck of a vascular aneurysm, and specifically across the neck of a cerebrovascular aneurysm. The barrier is a stent or neck bridge that completely or partially blocks the flow of blood into the aneurysm and, further, prevents the migration of embolic coils out of the aneurysm and into the parent vessel. The neck bridge or stent of the present invention comprises elements for superior flexibility and stability when placed within the parent vessel. The neck bridge or stent of the present invention is loaded into the catheter by either being rolled tightly and sheathed or stretched to permit loading into the delivery catheter in a small, highly flexible configuration that may be advanced through the cerebrovasculature to pathological aneurysms.

### FIELD OF THE INVENTION

This invention relates to generally to methods and devices for medical treatment and more particularly to methods and devices for treating defects (e.g., aneurysms, fistulas, aberrant branch vessels and arterio-venous malformations) that occur in blood vessels and other luminal anatomical structures.

### BACKGROUND OF THE INVENTION

Aneurysms are a common defect in the vascular system. Aneurysms are generally asymptomatic until they rupture; in which case, the effects are severe and often fatal due either to exsanguinations or penumbral damage to tissue near the aneurysm. The effects of a ruptured cerebrovascular aneurysm are those of stroke and include death, loss of sight, loss of hearing, loss of balance, loss of the use of muscles on one or both sides of the body. However, prior to rupture, aneurysms may present with mass effect or as a palpable structure within the body. Currently, treatment of cerebrovascular aneurysms, or aneurysms within the brain, are typically accomplished using either open surgical techniques or endovascular techniques. Open surgical techniques, which were developed first, require cutting through the skin and skull bone and moving aside brain material so that the aneurysm may be clipped or sutured and excised. These techniques entail high risk and are performed only when absolutely necessary because of the high rates of mortality and morbidity associated with such open surgical procedures.

The high operative mortality and morbidity of surgical clipping led to the search for alternatives, of which endovascular approaches to aneurysm repair are currently being developed. Endovascular and percutaneous placement of catheters to treat malformations and aneurysms of the cerebrovasculature entail lower risk of morbidity and mortality than surgical approaches but the long-term efficacy of endovascular approaches is still being evaluated. Aneurysms of the vasculature are treated today with stents, grafts, stent-grafts and embolic materials placed by endovascular techniques. These stents, grafts and stent-grafts serve to wall-off or isolate the aneurysm from the systemic blood pressure, the continued exposure to which will cause eventual rupture of the aneurysm. The through lumen of the vessel is, theoretically, kept patent so that the vessel can continue to function to deliver blood flow to distal vasculature. Embolic materials have been shown to exhibit utility in treating aneurysms of the brain. These brain or cerebrovascular aneurysms are generally small and have a sac-shape with a narrowed neck so that they look somewhat like a berry. These cerebrovascular aneurysms are currently filled with embolic materials such as platinum metal coils. The coils are, typically, delivered endovascularly by catheters inserted through the femoral artery. The first coils used to embolize the vasculature were tried in the 1970's (Gianturco et al., Mechanical Devices for Arterial Occlusions, 124 Am. J. Roent. 428 (1975) to embolize the renal arteries. Guglielmi et al., working with Target Therapeutics, Inc. developed an electrolytically detachable platinum coil, called the Guglielmi Detachable Coil that has proven beneficial in embolizing cerebrovascular aneurysms. An early citation on the use of the Guglielmi Detachable Coil (GDC) is Casasco, et al., Selective Endovascular Treatment of 71 Intracranial Aneurysms with Platinum Coils, 79 J. Neurosurgery 3 (1993). The use of platinum coils entails packing or stuffing the aneurysm with sufficient coils that the sac of the aneurysm is protected by the coil mass and the thrombus that forms therein.

Cerebrovascular aneurysms are clearly located in a critical area of the body. Any dislodgement or migration of a coil or incomplete packing of the aneurysm so that the sac wall is exposed to arterial pressure could have catastrophic results to the patient. Death and stroke leading to neurological impairment is not an uncommon result of coil migration. Such dislodgement or migration of embolic coils is a commonplace event. Although retrieval is sometimes possible, the retrieval procedure is not without complications similar to those of coil migration.

Embolic coils such as the GDC are more stable in aneurysms that have a sac diameter twice that of the neck separating the sac from the parent blood vessel. However, a large number of aneurysms do not have a small neck. Many aneurysms have a neck diameter equal to that of the sac and these are termed “wide neck” aneurysms. Another group of aneurysms have a neck width greater than that of the aneurysm sac. Yet another group of aneurysms, termed “fusiform” have no sac shape but are rather characterized by a widening of the blood vessel around most, or all, of its circumference. Aortic aneurysms are generally of the fusiform configuration.

Embolic coils will not remain placed in a fusiform aneurysm or an aneurysm with a neck greater in diameter than that of the sac. Newer coils allow stable placement in wide neck aneurysms but the older GDC devices often migrate from wide neck aneurysms. In addition, embolic materials fabricated from polymeric materials that solidify upon placement will migrate even more aggressively than coils and may not remain in place easily in aneurysms with small necks.

There is a need for improved devices to facilitate packing cerebrovascular aneurysms in patients. Aneurysms with wide necks or fusiform configuration are especially problematic. Some method of maintaining coverage over the neck of the aneurysm is required to either isolate the aneurysm or to retain embolic material within the aneurysm so that it will not migrate. Such devices have been termed “neck bridges”. The use of standard stents to cover the neck of an aneurysm is inappropriate since standard stents are too inflexible to be delivered endovascularly to the cerebrovasculature. Most aneurysms occur at the level of the Circle of Willis or even more distally. Endovascular access to the Circle of Willis is attained through the vertebral arteries or the carotid siphons, both of which are highly tortuous and prevent all but the most flexible of devices to pass. Another issue with prior stents, grafts, and stent-grafts is that they provide too much coverage within the parent vessel. Small, but vital, feeder vessels often lead from the parent vessel. Preventing blood flow into one or more of these feeder vessels has the potential of causing significant neurological dysfunction. Thus, any device located in the parent vessel must have minimal wall coverage so as to have a minimal chance of blocking a feeder vessel. Devices of the prior art designed to be sufficiently flexible on delivery to pass into the Circle of Willis or beyond are generally unstable upon deployment and become distorted, thus increasing the risk of migration downstream or generating emboli.

### SUMMARY OF THE INVENTION

The present invention is an improvement on stents or neck bridges of the prior art in that it provides for high stability in the implanted configuration. In addition, the present invention is collapsible into a sufficiently small delivery profile as to be able to be delivered into the Circle of Willis or beyond. In the delivery configuration, the stent of the present invention is highly flexible. In one embodiment, the stent retains constant length during delivery, deployment and after detachment. This embodiment of the stent is beneficial because guesswork and clairvoyance are not required in order to determine the final deployed length of the stent.

In another embodiment, the stent is stretched longitudinally when loaded into the delivery catheter, thus permitting extremely small delivery profile and high delivery flexibility. This configuration, however, leads to stent length changes between the delivery and deployed configurations. An advantage of this configuration is that, following deployment, the stent may be recaptured within the delivery catheter and re-deployed multiple times, prior to detachment from the delivery system.

The stent of the present invention is an axially elongate structure, comprising a series of circumferential rings connected by longitudinally projecting connecting members. The rings are incomplete in that the overall appearance of the stent is that of a ribcage. The configuration of the stent leads to very high stability when deployed in a cerebrovascular blood vessel. In the preferred embodiment, the longitudinally projecting members are configured as a “V” or in a notch. The notch or “V” configuration improves flexibility of the connecting members. Depending on the cross-sectional configuration of the connecting members, with square being ideal, the notching imparts improved flexibility in multiple degrees of freedom. The circumferential rings, struts or bars are, in another embodiment, disposed at an angle rather than perpendicular to the axis of the stent. Thus, the rings may be canted at an angle other than 90 degrees from the axis of the stent or they may form a spiral structure.

In yet another embodiment of the invention, the circumferential rings near the center of the axially elongate structure are axially thicker than those rings closer to the ends of the structure. In yet another embodiment of the invention, the circumferential rings near the center are more closely spaced than those at the ends of the structure. In yet another embodiment of the invention, the circumferential rings near the center of the axially elongate structure are wider toward one side than their width on the other side and wider than the rings at the ends of the axially elongate structure. In yet another embodiment of the invention, the rings are incomplete and the longitudinally projecting members form a continuous spine, preferably with notching. In this embodiment, the incomplete rings appear as the teeth on a comb.

In yet another embodiment of the invention, the stent is fabricated using laser etching. The laser is used to etch a metal tube or flat sheet to form the shape. A computer numerically controlled stage is used to allow for complex machining in a repeatable manner as is required to fabricate the complex shape of the stent.

In yet another embodiment of the invention, the stent is fabricated using photochemical etching. The pattern is etched out on a flat sheet of material. Following the photochemical etching process, the flat pattern created by the photoetching process is formed into a rolled tubular configuration. This rolled tubular configuration is optionally heat set into shape using a sand bath, salt bath, oven or other heat-treating system. In yet another embodiment of the invention, the stent is fabricated using electrochemical discharge machining (EDM). A flat sheet of material or tubular material is suitable for the EDM process. In yet another embodiment of the invention, the stent is fabricated using any of the aforementioned manufacturing processes on a flat sheet of material. The machining pattern is a distorted pattern that is rendered undistorted by bending the flat sheet into a tubular axially elongate structure. The exact machining pattern is determined by machining an axially elongate structure in the preferred compressed configuration and then bending the axially elongate structure into a flat sheet. The resulting pattern of openings describes the preferred machining pattern.

The stent of the present invention is, preferably, fabricated from shape memory metals such as nickel titanium alloys. Such nickel titanium alloys are called nitinol. Nitinol, under certain conditions, possess pseudoelastic or superelastic properties. They also exhibit characteristics such as shape-memory. Shape-memory properties are activated by temperature changes. The shape-memory property allows the stent to be cooled and loaded within the delivery catheter in a low-stress martensitic condition. When the stent is exposed to the temperatures of the body's cardiovascular system, the stent will become austenitic and assume a pre-determined configuration, in this case expanded to the desired implant configuration. Other materials suitable for stent fabrication include cobalt nickel alloys such as Stellite 21, Elgiloy, MP-35N and the like.

The stent of the present invention is, preferably, coated with anti-thrombogenic agents such as covalently or ionically bonded heparin. Such coatings are selectively applied only on the interior and interspaces between the stent members. The exterior of the stent, especially, in the high-density region near the center of the axially elongate structure are preferably not coated with anti-thrombogenic agents. These central regions are, in another embodiment, coated with thrombogenic agents designed to encourage thrombosis. Such thrombogenic agents include protamine sulfate.

The stent of the present invention is, preferably, coated with radiopacity enhancing materials. This is desirable since nitinol is not highly radiodense in the quantities used to form a cerebrovascular stent. Some method of enhancing radiopacity is desirable. The use of platinum, tantalum, gold or other markers adhered to the stent is desirable. In another embodiment, the nitinol stent is vapor deposition coated with tantalum, gold, platinum or the like.

In another embodiment of the invention, the stent is compressed into a rolled configuration prior to insertion into the delivery catheter. The stent compression apparatus is an axially elongate structure with a series of projections like a comb. The projections are rotated circumferentially, grabbing the connector bars between stent ribs and rolling the stent into a small diameter. In this small diameter, an exterior shield is advanced over the stent and the projections are retracted. The shielded stent is, next, loaded into the delivery catheter where the catheter constrains the ribs. The constraint is, preferably, an axially elongate flexible sheath that is withdrawn, relative to more proximal components of the delivery catheter, to deploy the stent.

In yet another embodiment of the invention, the stent is loaded over a rotational collar with projections, hooks or slots, which engage with features on the stent. The rotational collar is rotated about its axis causing the stent to roll down and compress radially over the collar. The rotational collar, in this embodiment, is integral to the delivery catheter and is used to wind the stent to its delivery diameter or unwind the stent to its deployed diameter. This system allows the stent to be deployed and retrieved multiple times if initial placement is unsatisfactory. Following satisfactory placement, the stent is released by overwinding the rotational collar, dissolving a link, pulling an attachment wire or opening a mechanical jaw.

In yet another embodiment, the stent is fabricated from wire, either round wire, oval wire, triangular wire, trapezoidal wire, or flat wire. The wire is formed into an axially elongate coil structure that is aligned with its major, or longitudinal, axis parallel to the parent vessel. The coil is formed with its individual loops spaced evenly and the outer diameter of the coil is equal to or slightly larger than that of the parent vessel inner diameter. In another embodiment, the coil windings are spaced more widely at the center of the axially elongate structure than toward the ends, thus increasing the density of the coils toward the longitudinal center and decreasing the density of the coils at the longitudinal ends of the axially elongate stent. The increased density of the coils at the center are beneficial for occluding the neck of an aneurysm while the decreased density of the coils toward the ends provide for stabilization within the parent vessel but minimized risk of feeder vessel occlusion. The stent is delivered within a catheter by stretching the coils out into a single, or double, long strand that is delivered as a wire, thus maximizing flexibility of the system during delivery through tortuous cerebrovasculature.

In yet a further embodiment of the coil stent, the stent is formed as a double helix. The double helix is, preferably, counterwound and wire crossings occur at intervals throughout the length of the stent. The counterwound coils offer the advantage of stretch resistance once the stent has been deployed. The counterwound double helix is, in a preferred embodiment, fabricated from a multi-filar structure to increase surface area and decrease the overall vessel occlusion of any given filament of the stent. The double helix is, preferably, fabricated from two completely separate coils that are separately actuated, although a double helix fabricated from a single strand that is folded back on itself is also functional. The separate double helix requires a delivery system that separately holds and winds down the separate coils to allow for control during delivery, deployment, and release. As in all of the embodiments of the stent cited in this invention, and in both the single helix and the double helix embodiments of the stent, the stent is attached to its delivery catheter using either a fusible link, mechanical jaws, or friction attachment. The friction attachment and the mechanical jaws are opened using a mechanical pusher (or pulling) wire, hydraulic pressure, or nitinol micro-actuator. The fusible link is actuated by electrolytic degradation of the fusible link or by melting of a polymer link by heat energy. The fusible link may also be detached through cryogenics to cause brittleness of the link, which is then moved slightly to crack the link and cause detachment.

The stent is releasably attached to the delivery catheter so that it is deployed and controlled until it is desired to release the stent. At this point, the stent is released. Release mechanisms suitable for this invention include mechanically openable jaws, meltable or dissolvable links and the like. The preferred release mechanism is a simple openable jaw that is actuated by a mechanical rod from the proximal end of the catheter or by a nitinol actuator that opens the jaws by application of electrical energy and heating to cause the jaws to open.

Yet another aspect of the invention is the method of implanting the stent and treating an aneurysm. The aneurysm is accessed endovascularly by guidewire and microcatheter access. The entry point to the patient is, preferably, the femoral artery. The guidewire(s), guide catheter, and microcatheter are routed retrograde up the aorta and into the carotid artery. Access is further enabled by traversing the carotid artery, through the carotid siphon, and into the Circle of Willis. Certain cerebrovascular locations are, preferably, accessed by the aorta and into the vertebral arteries. The basilar tip, a common location for aneurysms, is preferably accessed through the vertebral arteries. The access is, preferably, monitored and guided through the use of fluoroscopy. Radiographic dye injection, angiography, roadmapping, and even magnetic resonance angiography are all useful tools for monitoring and guiding catheter access to the cerebrovasculature. The typical fluoroscopic system preferable for this type of access is a biplanar system that allows viewing in two roughly orthogonal directions. Radiographic dye injection and fluoroscopy are performed to verify aneurysm dimensions, configuration and treatability.

The stent is, preferably, preloaded into its delivery catheter and sterilized prior to delivery to the catheterization laboratory in a single or double aseptic package. The stent and delivery catheter are removed from their packaging and routed either over a guidewire or through a guiding catheter, which were pre-positioned at the desired location within the aneurysm. The stent and its delivery catheter are advanced to the location of the aneurysm. The distal end of the stent is located fluoroscopically at the desired location anatomically distal to the aneurysm. The stent is advanced or deployed out of its delivery catheter so that it now forms a partial barrier across the aneurysm neck and its proximal end is located anatomically proximal to the aneurysm. Special effort is made to avoid occlusion of feeder vessels through fluoroscopic analysis. Rotation of the stent is performed, if required to achieve proper circumferential alignment. Retraction and redeployment or forced movement of the stent are used to longitudinally adjust the stent within the parent vessel of the aneurysm. Once the correct location is verified, the stent is detached from the delivery catheter. Embolizing materials such as platinum coils and/or polymeric materials are, next, injected or inserted into the aneurysm using standard endovascular techniques. Access to the aneurysm is, preferably, made through spaces between the structural members of the initially inserted neck-bridge stent. The microcatheter or guidewire followed by microcatheter are advanced within the neck bridge and then advanced laterally through the neck bridge structure to reach the aneurysm sac.

In yet another aspect of the invention, an embolic coil is disclosed that is deliverable through the neck bridge stent. Currently available coils include platinum devices manufactured by Target Therapeutics, MicroVention, Inc. J&J Cordis and Micrus. This improved coil is a series of loops joined tangentially. The loops are, preferably metallic in construction with such materials as nitinol and inconel being preferred materials. The device is configured with between one and ten large wire loops and between one and ten smaller wire loops. These smaller wire loops are configured at the proximal end of the structure nearest the attachment point to the delivery catheter. Radiopaque markers fabricated from materials such as platinum, platinum-iridium alloy, gold, tantalum and the like. These markers are approximately 0.010 to 0.030 inches long and are preferably located at least at the ends of the structure but even more preferably one marker is located on each loop. When deployed, the large loops fill the aneurysm and are oriented in planes that are disposed at an angular displacement from the plane of adjacent loops. The small loops reside at the neck of the aneurysm and open to a flower petal shape to assist in blocking the neck of the aneurysm. Such neck blockage minimizes blood flow impingement into the aneurysm and assist in retaining additional coils or embolic materials that are deployed within the aneurysm. In an additional embodiment of the invention, all or part of the wire form structure is coated with Thrombogenic materials such as prothrombin or protamine sulfate. All or part of the invention is, preferably, coated with hydrophilic hydrogels or sponge materials to provide additional filling within the aneurysm.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1A illustrates a side view of a fully expanded stent comprising a plurality of hoops and a plurality of longitudinal connecting bars, according to aspects of an embodiment of the invention;

FIG. 1B illustrates a side view of a stent, fully compressed by rolling, not stretching, comprising a plurality of hoops and a plurality of longitudinal connecting bars, according to aspects of an embodiment of the invention.

FIG. 2A illustrates a side view of a fully expanded stent comprising a plurality of hoops and a plurality of longitudinal connecting bars, wherein the longitudinal connecting bars are “V” shaped or notched, according to aspects of an embodiment of the invention;

FIG. 2B illustrates a side view of a stent, fully compressed by rolling, not stretching, comprising a plurality of hoops and a plurality of longitudinal connecting bars, wherein the longitudinal connecting bars are “V” shaped or notched, according to aspects of an embodiment of the invention;

FIG. 3A illustrates a side view of a stent, comprising a plurality of hoops and a plurality of longitudinal connecting bars, partially loaded into a delivery catheter by means of stretching, according to aspects of an embodiment of the invention;

FIG. 3B illustrates a side view of a stent, comprising a plurality of hoops and a plurality of longitudinal connecting bars, and delivery catheter with the stent fully loaded into the delivery catheter, according to aspects of an embodiment of the invention;

FIG. 4A illustrates a side view of a stent, comprising a plurality of hoops and a plurality of longitudinal connecting bars, wherein the hoops near the center of the stent are spaced closer than are the hoops near the ends of the stent, according to aspects of an embodiment of the invention;

FIG. 4B illustrates a side view of an expanded stent, comprising a plurality of hoops and a plurality of longitudinal connecting bars, wherein the hoops near the center of the stent are longitudinally wider than are the hoops near the ends of the stent, according to aspects of an embodiment of the invention;

FIG. 4C illustrates a side view of an expanded stent, comprising a plurality of hoops and a plurality of longitudinal interconnecting bars, wherein the hoops near the center of the stent are longitudinally wider than are the hoops near the ends of the stent, and, further, wherein the hoops near the center of the stent are spaced more closely than are the hoops near the ends of the stent, according to aspects of an embodiment of the invention.

FIG. 5A illustrates a side view of a distal tip of a delivery catheter, configured to roll a stent into a delivery diameter smaller than its fully expanded diameter, according to aspects of an embodiment of the invention;

FIG. 5B illustrates a side view of a distal tip of a delivery catheter, comprising an element to roll or wind a stent into a diameter smaller than its fully expanded diameter, and a stent, which is beginning to become wound upon said distal tip of the delivery catheter, according to aspects of an embodiment of the invention.

FIG. 5C illustrates the proximal end of a delivery catheter, configured to roll a stent into a delivery diameter smaller than its fully expanded diameter, according to aspects of an embodiment of the invention;

FIG. 6A illustrates a cerebrovascular aneurysm with a narrow neck, suitable for embolizing with coils, according to aspects of an embodiment of the invention;

FIG. 6B illustrates a cerebrovascular aneurysm with a wide neck, which provides inadequate resistance to migration for the coils being implanted, according to aspects of an embodiment of the invention;

FIG. 6C illustrates a wide neck cerebrovascular aneurysm, with an expanded stent of the present invention placed within the parent vessel and across the neck of said aneurysm such that embolic coils may be safely placed with minimal risk of migration, according to aspects of an embodiment of the invention;

FIG. 7A illustrates an expanded stent comprising two separate counterwound helical coils that resist stretching once the stent has been deployed, the coil further comprising multiple filaments, according to aspects of an embodiment of the invention;

FIG. 7B illustrates an expanded stent comprising a single length of counterwound helical coil that resists stretching once the stent has been deployed, the coil further comprising multiple filaments, according to aspects of an embodiment of the invention.

FIG. 8 illustrates a cerebrovascular aneurysm near a bifurcation with a stent placed within the parent vessel and across the neck of the aneurysm, wherein feeder vessels are avoided by minimizing material mass near the ends of the stent, according to aspects of an embodiment of the invention.

FIG. 9A illustrates a stent with a longitudinal strut that is in its shortened Z-folded configuration, capable of elongation under the influence of increased temperature, according to aspects of an embodiment of the invention.

FIG. 9B illustrates the stent of FIG. 9A with the Z-folded longitudinal strut in its expanded, unfolded configuration, according to aspects of an embodiment of the invention.

### DETAILED DESCRIPTION OF THE INVENTION

In accordance with one or more embodiments of the present invention, a stent or neck bridge for assisting with embolization of cerebrovascular and other aneurysms is described herein. In order to fully specify this preferred design, various embodiment specific details are set forth, such as the materials, configuration of the stent, methods of stent loading into the catheter, and deployment of the stent. It should be understood, however that these details are provided only to illustrate the presented embodiments, and are not intended to limit the scope of the present invention.

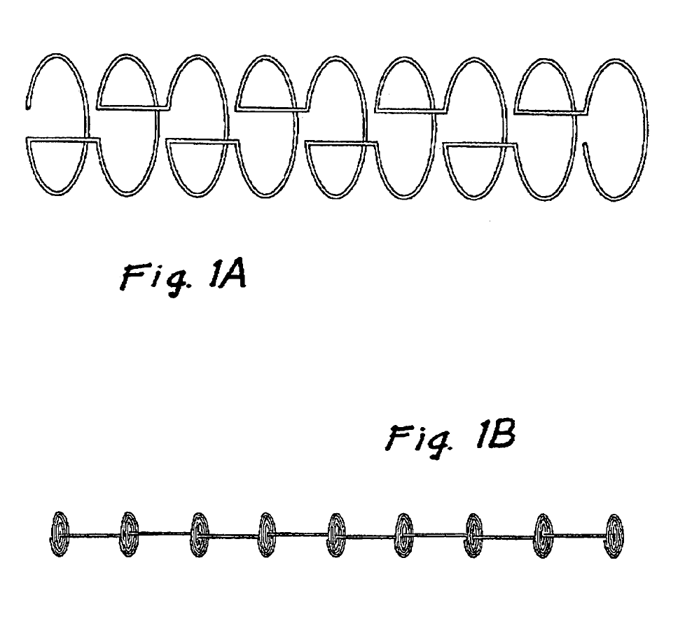

FIG. 1A illustrates a side view of a stent 10 of the present invention comprising a plurality of circumferential bars 12, a plurality of connecting bars 14, and a stent end-connector 20.

The circumferential bars 12 form incomplete rings or hoops, each of which is terminated with a connecting bar 14. The stent end-connector 20 is affixed to either one connecting bar 14 or one circumferential bar 12.

The circumferential bars 12, the connecting bars 14 and the end connector 20 are all, preferably fabricated from the same materials. Materials suitable for fabricating these components of stent 10 include, but are not limited to, platinum, platinum-iridium, tantalum, tin, gold, nitinol, Elgiloy, stainless steel, titanium, MP-35N, other cobalt-nickel alloys, polymers such as PET, polylactic acid, and polyglycolic acid, and the like. In order to give the stent 10 improved radiopacity, the components may be coated with platinum, tantalum, gold, platinum-iridium and the like. The coating process is typically a vapor deposition process but a dipping process is appropriate for certain materials.

The stent 10 is preferably fabricated from materials that exhibit spring resilience such as nitinol, Elgiloy, or spring stainless steel (types 304 and 416, for example). In this way, the stent 10 is expandable without the need for a balloon catheter to malleably expand the stent.

Fabrication techniques for the stent 10 include, but are not limited to, electron discharge machining (EDM), photochemical etching, laser etching, conventional machining, wire drawing, spring winding, and the like.

The expanded outside diameter of the stent 10 ranges from 2.0 mm to 10 mm and, more preferably, from 3 mm to 5 mm. The length of the stent 10 ranges between 5 mm and 100 cm and, more preferably, the stent length ranges between 10 mm and 50 mm. The radial thickness of the circumferential bars 12 and the connecting bars 14 ranges between 0.0001 inches and 0.010 inches. More preferably, the stent 10 thickness ranges between 0.00015 inches to 0.003 inches and most preferably between 0.0002 and 0.001 inches.

The material that makes up the circumferential bars 12 and the connecting bars 14 is round, oval, triangular, trapezoidal, or rectangular in cross-section. The triangular, trapezoidal, and rectangular cross-sectional versions preferably are slightly rounded at the edges to minimize the risk of tissue damage that could lead to hyperplasia.

The stent end-connector 20 is an enlarged region at the end of the structure closest to the stent delivery catheter. The stent end-connector 20 is designed for attachment to a stent delivery mechanism such as, but not limited to, a pair of jaws, a dissolvable coupling, a friction coupling, a hydraulically expanding coupling, a hydraulically lengthening or shortening coupling, a shape memory actuator operated coupling, a hydraulically compressive coupling, and the like. The friction coupling is uncoupled by application of hydraulic pressure at the proximal end of the catheter. The hydraulic pressure is transmitted along the length of the catheter and acts at the distal end of the catheter to overcome the friction of the coupler and detach the stent 10

In yet another embodiment of the stent 10 of FIG. 1A, the circumferential bars 12 are not disposed perfectly orthogonal to the axis of the stent 10 but are disposed at an angle other than orthogonal to the longitudinal axis of the stent 10. An exemplary configuration is that of a spiral or partial spiral where one side of the circumferential bar 12 is closer to the end of the stent 10 than is the other side of the circumferential bar 12.

FIG. 1B shows another embodiment of the stent 10 of FIG. 1A comprising the circumferential bars 12, the connecting bars 14 and the stent end-connector 20. The stent 10 shown in FIG. 1B is in its compressed or delivery configuration such that no length extension has occurred. The stent 10 compression is accomplished by rolling the stent 10 so that the connecting bars 14 joining opposite sides of the circumferential bars 12 are initially pulled toward each other and the wrapping or rolling continues until a minimum radial dimension has been achieved. This configuration of eliminating length extension during compression of the stent 10 is important in that the operator can accurately place the stent without fear of occluding structures that should be left unoccluded or fear of missing an important occlusion area such as an aneurysm neck. The compressed or delivery diameter of the stent 10 is typically less than 0.040 inches and, preferably, less than 0.020 inches.

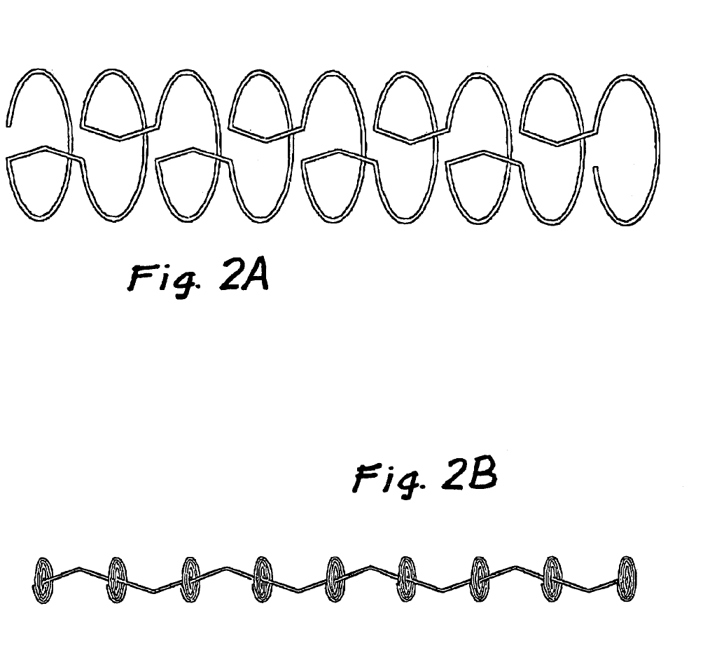

FIG. 2A illustrates another embodiment of the stent 10 comprising a plurality of circumferential bars 12, a plurality of connecting bars 14, and a stent end-connector 20. In this embodiment, the connecting bars 14 further comprise a bend or notch 16 that enhances flexibility of the connecting bar and, in consequence, the stent 10. Such flexibility is especially important in a cerebrovascular stent because of the need to deliver the stent 10 through a tortuous pathway such as the carotid siphon. Linear flexibility of the stent 10 is also important in the expanded, or implanted, configuration because the vessels in which the stent 10 is implanted are often curved and not straight. The notch 16 or bend allows flexing to occur around an axis different from that of a straight connecting bar 14.

FIG. 2B illustrates the stent 10 of FIG. 2A compressed into its delivery diameter. The compression has occurred so that the stent 10 has not substantially changed is overall length even though the outer diameter of the stent 10 has been reduced substantially. The compressed diameter of the stent 10 is less than 0.040 inches and, preferably, less than 0.020 inches.

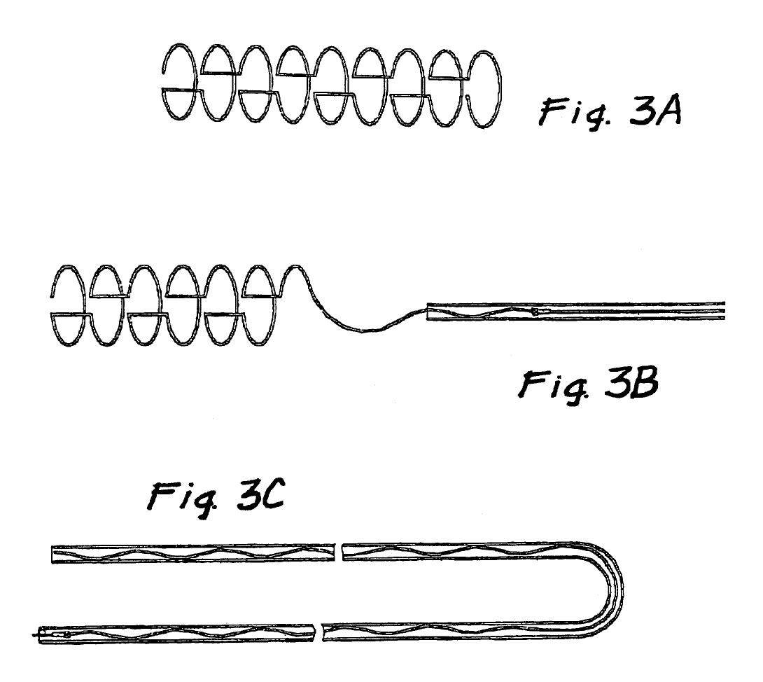

FIG. 3A illustrates yet another embodiment of the stent 10 wherein the stent 10 is being withdrawn into a delivery catheter tube 18. The stent 10, in this embodiment, further comprises a plurality of circumferential bars 12, a plurality of connecting bars 14, a deformed bar section 16, a stent end-connector 20, a catheter connector 22, a set of jaws 24, and a control rod 26. In this embodiment, the stent 10 is of the same configuration as that shown in FIG. 1A, except that it is collapsed into its delivery configuration by deforming the circumferential bars 12 and the connecting bars 14 into a relatively linear deformed bar 16 configuration. The stent end-connector 20 is mated to the openable catheter connector 22, further comprising openable jaws 24 and a control rod 26. The control rod 26 is affixed to the catheter connector 22 and is able to pull or push the catheter connector 22 along the axis of the catheter tubing 18. The control rod 26 further comprises a mechanical or electrical linkage (not shown) that operates the jaws 24. The jaws 24 are opened an closed by mechanical actuation of the linkage, or by electromagnetic force, or by activation of a shape memory actuator by electrical Ohmic heating.

FIG. 3B illustrates the stent 10 of FIG. 3A after it has been fully withdrawn within the catheter tubing 18. The compressed stent 10 is, in this embodiment, a length of fully deformed wire 16 that is terminated by a stent connector 20 and releasably connected to the control rod 26 by the catheter connector. The stent 10 is stretched into a generally longitudinal configuration, which possesses maximum possible flexibility and minimum possible delivery profile. The single length of deformed wire 16 is maximally flexible for delivery and is able to fit into a very small delivery diameter of 0.010 inches or less. The deformed wire 16 has a radial dimension of less than 0.010 inches and preferably, less than 0.005 inches. The circumferential bars 12, the connecting bars 14 and the deformed wire 16 configuration have the same cross-sectional characteristics as those of the stent 10 of FIG. 1A. Tensile forces on the control rod 26 cause withdrawal of the stent 10 into the catheter tubing 18 while compressive forces on the control rod 26 cause deployment of the stent 10 out the end of the catheter tubing 18.

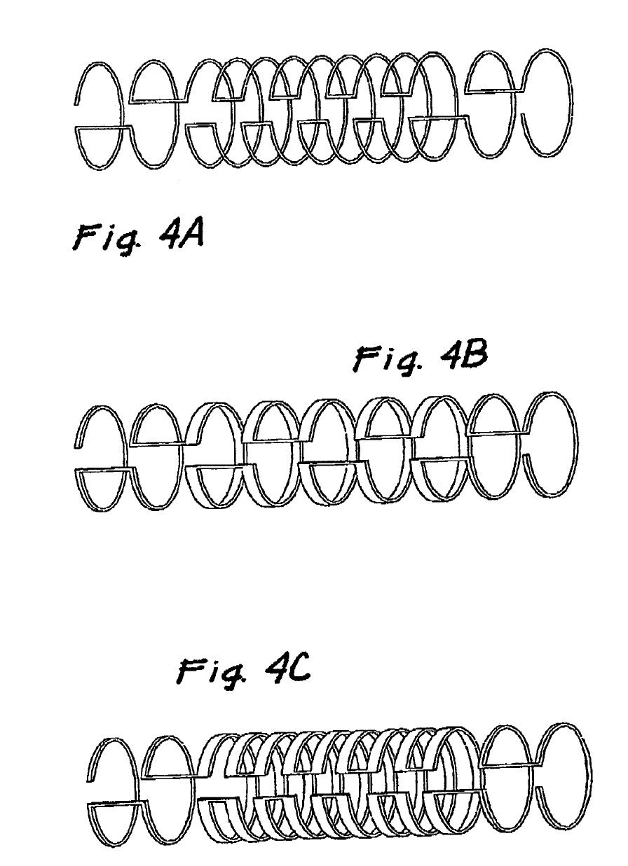

FIG. 4A illustrates yet another embodiment of a stent 10 of the present invention comprising a plurality of circumferential bars 12 and a plurality of connecting bars 14. The number of circumferential bars 12 per unit length of stent 10, near the center of the stent 10 is greater than the number of circumferential bars 12 per unit length of the stent 10 near the ends of the stent 10. The increased density of circumferential bars 12 near the center of the stent 10 facilitates occlusion of structures such as an aneurysm neck while minimizing occlusion of feeder vessels in regions where occlusion is not desired.

FIG. 4B illustrates yet another embodiment of a stent 10 of the present invention comprising a plurality of circumferential bars 12 and a plurality of connecting bars 14, wherein the circumferential bars 12 are axially larger and, optionally, the connecting bars 14 are circumferentially larger near the center of the stent 10 than they are at the ends of the stent 10. This configuration of the stent 10 facilitates occlusion of structures such as an aneurysm neck while minimizing occlusion of feeder vessels in regions where occlusion is not desired.

FIG. 4C illustrates yet another embodiment of a stent 10 of the present invention comprising a plurality of circumferential bars 12 and a plurality of connecting bars 14, wherein the circumferential bars 12 are axially larger and, optionally, the connecting bars 14 are circumferentially larger near the center of the stent 10 than they are at the ends of the stent 10. In addition, the circumferential bars 12 are spaced closer together near the center of the stent 10, than is their spacing near the ends of the stent 10. By combining these two features of bar wideness and density, maximum occlusion of a structure near the center of the stent 10, such as an aneurysm neck, and minimum occlusion of feeder vessels or other structures near the ends of the stent 10 are minimized.

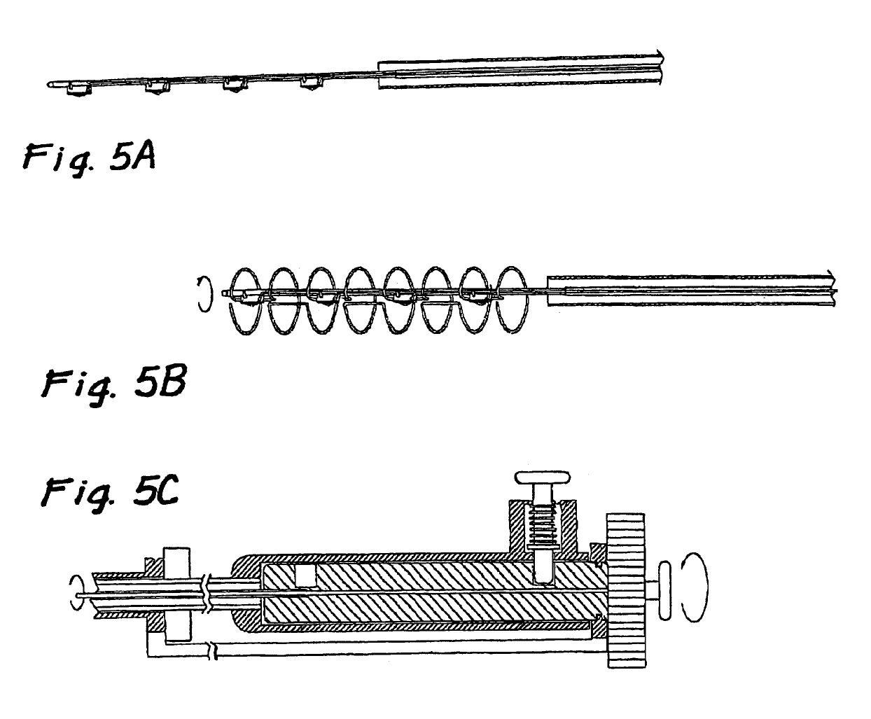

FIG. 5A illustrates the distal tip of a delivery catheter 31 of the present invention, further comprising a length of axially elongate catheter tubing 18, an axially elongate control rod 26, an axially elongate stent winding bar 30, a plurality of stent winding tabs 32, a plurality of stent winding tab holes 29, and a stent lock 33. The stent winding bar 30 is affixed to the proximal end of the control rod 26 and the stent winding tabs are permanently affixed to the stent winding bar 30 and project radially outward therefrom. The stent lock 33 is an axially elongate length of wire that slideably traverses the length of the delivery catheter 31 from its distal end to its proximal end within or along the control rod 26. At the distal end of the delivery catheter 31, the stent lock 33 wire forms multiple strands that lock through the plurality of holes 29 in the plurality of stent winding tabs 32. Preferably, there are two holes 29 in each stent winding tab 32. The stent lock 33 is slideably affixed, at the proximal end of the delivery catheter 31, through holes or fenestrations in the stent winding tabs 32. The stent lock 33, like the control rod 26, is actuated from the proximal end of the delivery catheter 31 by the physician.

The stent lock 33 is preferably a length of wire fabricated from materials such as, but not limited to, stainless steel, titanium, nitinol, cobalt nickel alloy, etc. The stent winding bar 30, the control rod 26, and the stent winding tabs 32 are preferably fabricated from materials such as, but not limited to, stainless steel, titanium, nitinol, cobalt nickel alloy, etc. The catheter tubing 18 is preferably fabricated from materials such as, but not limited to, PEBAX, wire wound PEBAX, braided wire reinforced PEBAX, polyurethane, polyethylene, polyamide, stainless steel wire coils, nitinol wire coils, and the like. The catheter tubing 18 is preferably thicker and stiffer at its proximal end than it is at its distal end. The catheter tubing 18, even more preferably, has graduated stiffness so that the stiffness decreases going from the proximal to the distal end of the delivery catheter 31.

FIG. 5B illustrates a stent 10 in the initial stages of winding upon the stent winding bar 30 of a delivery catheter 31. The delivery catheter 31 further comprises a stent lock 33, a control rod 26, a length of catheter tubing 18, and a plurality of stent winding tabs 32. The stent 10 further comprises a plurality of circumferential bars 12, a plurality of longitudinal connecting bars 14, and a plurality of counter-oriented connecting bars 15. The stent winding tabs 32 catch on the longitudinal connecting bars 14 and wind the stent 10 by acting on the longitudinal connecting bars 14. The individual strands of the stent lock 33 pass through holes in the stent winding tabs 32 and secure or trap the longitudinal connecting bars against the stent winding tabs 32. The control rod 26 and the stent winding bar 30 have the characteristics of longitudinal flexibility but also column strength, tensile strength, and torsional rigidity, otherwise known as torqueability. Continued rotation of the control rod 26 and the stent winding bar 30 causes the stent 10 to completely wind down against the stent winding bar 30 so as to generate the smallest possible delivery profile. When the stent 10 is completely wound down around the stent winding bar 30, the catheter tubing 18 is advanced to cover the stent 10 and prevent unwinding.

Referring to FIG. 5B, delivery of the stent 10 by the delivery catheter 31, once the tip of the delivery catheter 31 has been fluoroscopically placed at the appropriate location within the vasculature, the control rod 26, stent winding bar 30 and the stent 10 are fixed in place relative to the vasculature. The catheter tubing 18 is withdrawn, keeping the stent 10 anatomically fixed in place. The stent 10 expands or unwinds at this point. Following confirmation of position and repositioning of the stent 10 as necessary, the stent lock 33 is activated so that the wire strands are pulled out of the holes in the stent winding tabs 32. The stent 10 is released at this point and the catheter 31 is withdrawn from the vasculature.

Further referring to FIG. 5B, the stent winding tabs 32 actively wind the stent 10 by pushing on longitudinal connecting bars or struts 14. The stent winding tabs 32 push on only half of the longitudinal connecting bars 14, and those bars are aligned in one orientation or side relative to the other half of the connecting bars 14. The counter-oriented connecting bars 15 do not have stent winding tabs pushing thereon. In another embodiment of the invention, however, these counter-oriented connecting bars 15, as well as the proximal end and distal end of the stent circumferential bars 12, are affixed to clamping mechanisms or stationary posts to control their movement. Control over the movement of the counter-oriented connecting bars 15 and the stent 10 ends maximizes ability to reposition the stent prior to final release into the cerebrovasculature.

In yet a further embodiment of the invention, either all of, or at least, the proximal most and distal most stent winding tabs 32 are fabricated from highly radiopaque material so as to clearly identify the proximal and distal extents of the stent 10 prior to release. Such radiopaque materials include, but are not limited to, tantalum, platinum, gold, and platinum-iridium. The radiopaque materials are welded to the stent winding bar 30 or are supplied as coatings to features, such as the proximal and distal stent winding tabs 32, on the catheter 31 that describe the extents of the stent 10. The stent winding bar 30 may also be rendered radiopaque by the methods herein described as an alternative embodiment. By making the stent winding tabs 32 radiopaque, rotational orientation may be controlled and a non-rotationally or circumferentially uniform stent 10 may be deployed in order to maximize coverage of the aneurysm neck and minimize coverage of the parent vessel.

FIG. 5C illustrates a cross-sectional view of the proximal end of a delivery catheter 31 adapted to deploy the stent of the present invention. The delivery catheter 31 proximal end further comprises a length of catheter tubing 18, a control rod 26, a winding knob 34, a hub 36, a winding shaft 46, a stent lock handle 48, a stent lock 33, a locking pin 42, a lock lever 38, a lock spring 40, a lock housing 43, and a plurality of locking detents 44.

The proximal end of the catheter tubing 18 is permanently affixed to the distal end of the hub 36. The lock housing 43 is permanently affixed to the exterior of the hub 36 or is fabricated integral to the hub 36. The winding knob 34 is permanently affixed to the proximal end of the winding shaft 46. The winding shaft 46 is movably constrained by the interior of the hub 36 and is able to both rotate and move longitudinally within the hub 36. The locking detents 44 are holes or circumferential grooves in the winding shaft 46 capable of accepting insertion of the locking pin 42. The spring 40 is longitudinally trapped between the lock housing 43 and the locking pin 42. The spring is radially constrained by the locking pin 42, which slideably resides on the interior of the spring 40. The lock handle 48 is permanently affixed to the locking pin 42.

The spring 40 is compressed when the locking pin 42 is withdrawn out of the locking detent 44. When the locking pin 42 is withdrawn out of the locking detent 44, the winding shaft 46, the winding knob 34 and the control rod 26 may be moved relative to the hub and attached catheter tubing 18. Referring to FIGS. 5B and 5C, rotation of the winding knob 34 and the attached winding shaft 46, causes rotation of the control rod 26, the winding shaft 30 and the stent 10. It is preferable that the distal locking detent 44 is a circumferential groove rather than a hole to prevent the need for proper orientation to control the extents of movement or travel of the winding shaft 46 relative to the hub 36.

Referring to FIG. 5C, in yet a further embodiment of the proximal end of the delivery catheter 31, the winding shaft 46 is linearly connected to, but rotationally free to move relative to, rotational collar 43, which is affixed to a stabilizing bar 47 that is further affixed to a sheath 49 inserted into the patient's vasculature and extending exterior to the patient. The stabilizing bar 47 ensures that the control rod 26 remains in place relative to the patient's external anatomy into which the sheath is inserted. A Toughy-Borst, or rotational sealing, valve 41 is optionally comprised by the sheath 49. A connection between the stabilizing bar 47 and the rotational hub 36 optionally further comprises a mechanical advantage such as a pistol grip and trigger or threaded jack-screw to controllably move the hub 36 relative to the stabilizing bar 47 and the sheath 49.

All components at the proximal end of the delivery catheter 31 are fabricated from polymeric materials or metals with consideration being given to biocompatibility and smooth inter-operability of said components.

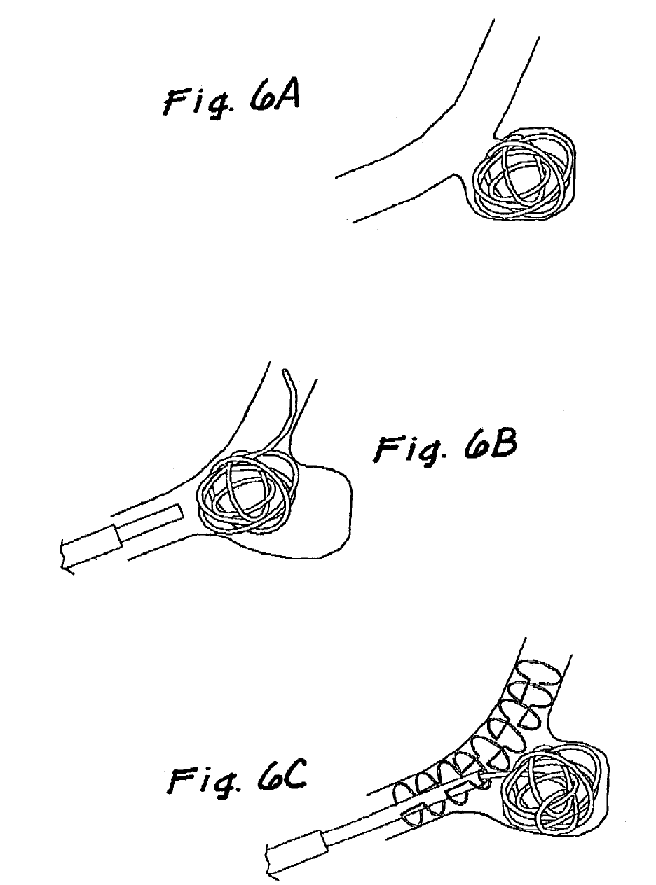

FIG. 6A illustrates a main or parent vessel 50 with an aneurysm 51 in the wall of the parent vessel 50. The aneurysm 51 further comprises an aneurysm sac 52 and an aneurysm dome or neck 54. A coil mass 56 has been endovascularly placed within the aneurysm sac 52 and is retained in place by the hoop strength of the coil mass 56 and the resistive forces exerted by the aneurysm neck 54. The aneurysm 51 is of the narrow neck type where the ratio of the dome diameter to the neck diameter is around 2:1. Narrow neck aneurysms 51 typically hold coil mass 56 easily and without a high risk of coil mass 56 migration. Typical coils used to create the coil mass 56 include the Guglielmi Detachable Coil (GDC), marketed by Boston Scientific, Inc., the MicroPlex Coil System (MCS), marketed by MicroVention, Inc. and the HydroCoil Embolization System (HES), marketed by MicroVention, Inc. Other embolic materials, such as polymeric materials delivered in solvents such as DMSO that leach out and permit solidifiying of the polymer are also used for aneurysm embolization.

FIG. 6B illustrates a parent vessel 50 comprising a side-wall aneurysm 51. The aneurysm 51 further comprises a sac or dome 52 and a neck 54. The neck 54 of the aneurysm 51 is nearly as wide as its dome with a dome to neck ratio of 1:1. A coil mass 56 is being deployed within the aneurysm by a catheter 62 and a guide catheter 60. The coil mass 56, further comprising a plurality of coil ends 64, is not adequately resisted by the neck 54 and the coil mass 56 has migrated into the lumen of the parent vessel 50. Such migration of the coil mass 56 causes parent vessel 50 occlusion or shedding of emboli that obstruct downstream vasculature and lead to stroke, if the vasculature is in the head. The occurrence of cerebrovascular emboli and subsequent stroke is often catastrophic, leading to conditions ranging from temporary memory impairment to permanent loss of motor function, or even cardiopulmonary arrest and death. Even the presence of a coil end 64 that migrates into the parent vessel 50 can cause catastrophic shedding of thromboemboli.

FIG. 6C illustrates a parent vessel 50 with an aneurysm 51 further comprising a sac or dome 52 and a neck 54. A coil mass 56 is being deployed within the aneurysm dome 52 through a delivery catheter 62 further placed through a guide catheter 60. The dome to neck ratio of the aneurysm in FIG. 6C is 1:1 or greater and the coil mass 56 is at high risk for migration out into the parent vessel 50. A stent 10 has been placed within the parent vessel 50 across the neck 54 to resist the force of the coil mass 56 trying to migrate out into the parent vessel 50. The stent 10 is generally placed first and the coil mass 56 is placed afterwards through openings in the stent 10 wall. In FIG. 6C, the coil mass end 64 is shown being deployed out of the end of the catheter 62.

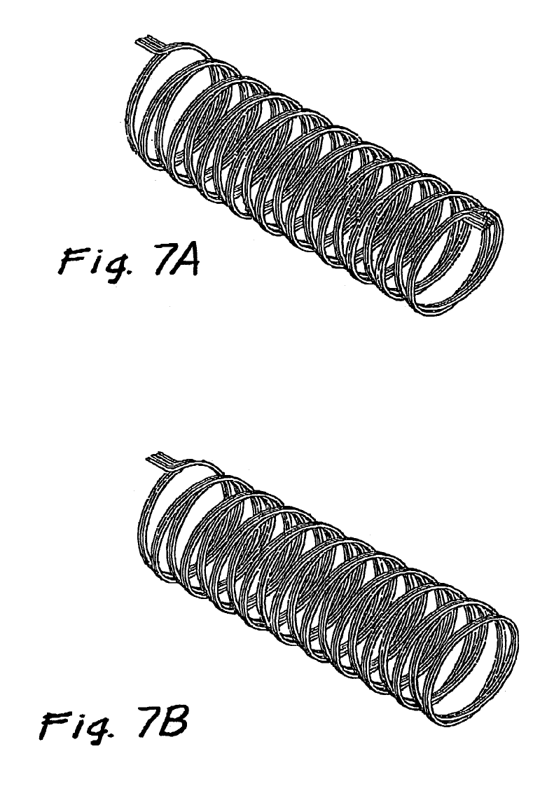

FIG. 7A illustrates a multi-filar stent 10 of the present invention. The stent 10 further comprises an outer helix 70 and an inner helix 72. The outer helix 70 and the inner helix 72 are two separate coil structures, each with a proximal end 76 and a distal end 78. In this embodiment, the inner helix 72 and the outer helix 70 are further comprised of multiple filaments of wire 74. In yet another embodiment, the inner helix 72 and the outer helix 70 are comprised of a single strand or monofilament of wire. This self-expanding elastomeric stent 10 is preferably fabricated from nitinol and further preferably comprises one or more radiopaque markers positioned, at least at the proximal ends 76 and the distal ends 78. This type of stent 10 has the advantage of being highly stable and resists deformation once placed. The stent is delivered by grabbing the distal end 78 and the proximal end 76 of the inner helix 72 and the outer helix 70 and rotating them in a direction counter to one another. The delivery system comprises this counter-rotating coupling and deployment system that can grab the stent 10 at four places.

In yet another embodiment of the invention, the delivery system is capable of not only counter rotating the proximal end 76 of the stent 10 but also of stretching and deforming the stent 10 to form a pair of long strands within the delivery catheter, a minimum delivery profile configuration. In this embodiment, the distal ends 78 of the stent 10 are grabbed by the connector of the stent delivery catheter and held immobile by that connector. A major advantage of the multi-filar construction, utilizing the multiple wire filaments 74, is that each of the filaments 74 may be coated with radiopaque materials such as, but not limited to, tantalum, gold, platinum, platinum-iridium and the like. Because the multiple filaments 74 each have a surface, the additive effect of the filament surfaces increases the amount of radiopaque material on the stent 10 and increases its visibility. The multi-filar construction is also beneficial in minimizing the occlusion of feeder vessels that exist within the cerebrovasculature as branches off the parent vessel.

FIG. 7B illustrates a stent 10 further comprising an outer helix 70 and an inner helix 72. The outer helix 70 and the inner helix 72 are fabricated from the same piece of wire, which is turned back on itself at the distal end 78 of the stent 10. The two helices form separate wire ends at the proximal end 76 of the stent 10. FIG. 7B illustrates a stent 10 fabricated from multiple filaments 74 of wire. The stent 10 may also be fabricated form a single wire element. This stent 10 is deployed by a delivery catheter that grabs the stent 10 only at the proximal end 76. The stent 10 is grabbed by a controllably openable coupler at each of the proximal wire ends 76. The two couplers are separately able to rotate counter to each other to wind the stent 10 down to a smaller diameter and then retract the stent within a sheath on the delivery catheter.

Referring to FIGS. 7A and 7B, the stent couplers on the delivery catheter are preferably openable jaws operated by mechanical linkage or electrical energy. The couplers may also be fusible links fabricated from electrolytically dissolvable metals or meltable polymers. The couplers may also be shape-memory actuator driven using ohmic heating and electrical power delivered from the proximal end of the delivery catheter. The couplers may also be hydraulically activated systems that pressurize a connection and force the stent out of that connection by hydraulic pressure.

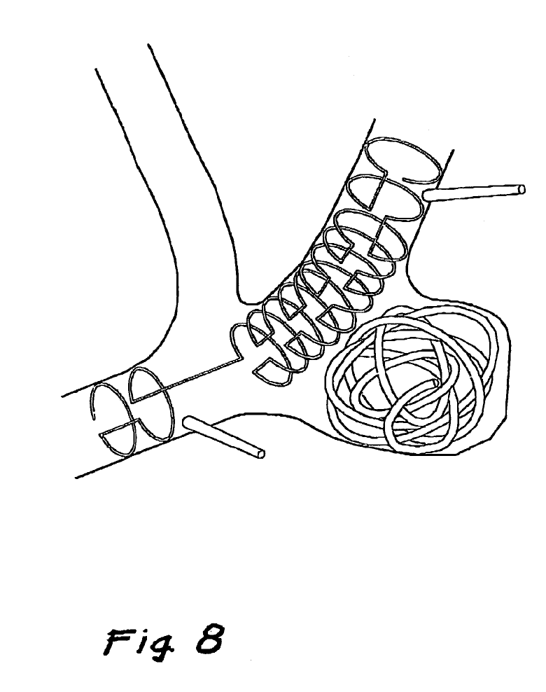

FIG. 8 illustrates a parent vessel 50 with a bifurcation further comprising a main branch vessel 58 and two small feeder vessels 66. The parent vessel further comprises an aneurysm 51, which further comprises an aneurysm neck 54 and an aneurysm dome or sac 52. The aneurysm sac is filled with an embolic coil mass 56. The embolic coil mass is held in the sac by a stent 10 placed across the neck 54 of the aneurysm 51. Referring to FIGS. 4A and 8, the stent 10 is configured with large gaps between the circumferential hoops 12 so as to allow unrestricted blood flow into the main branch vessel 58 and the feeder vessels 66. These large gaps are generated by extending the length of selected longitudinal struts 14 in the stent 10. Such tailoring is preferably performed prior to stent 10 implantation and this tailoring is based on careful roadmapping and analysis of the anatomical structure of the vasculature and the aneurysm.

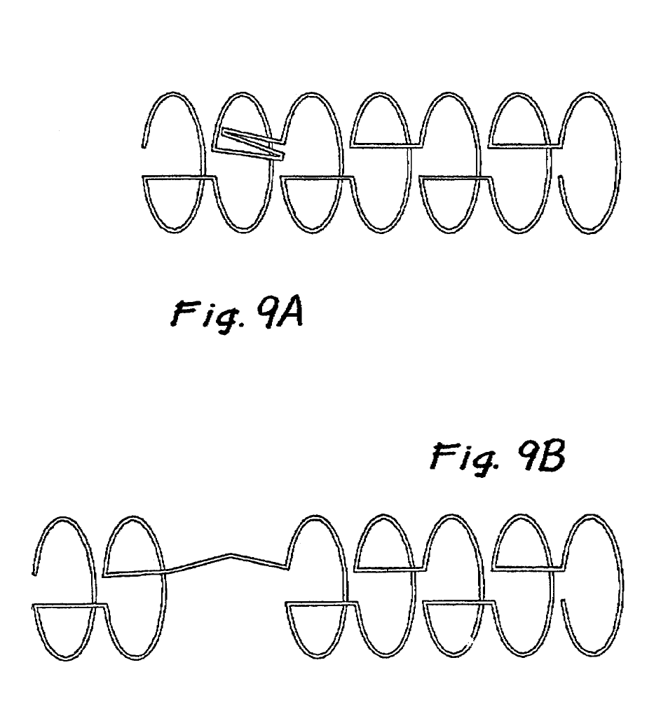

FIG. 9A illustrates yet another embodiment of the stent 10, which further comprises a plurality of circumferential struts 12 and a plurality of longitudinal struts 14 the latter of which may be modified in situ. Once a stent 10 has been placed, it may be required to readjust the position of the circumferential struts 12 to avoid obstructing a feeder vessel or branch vessel. These modifications, or circumferential strut 12 position readjustments, are accomplished using shape-memory longitudinal struts 14 that are Z-folded, distorted, or curved to reduce longitudinal length relative to the overall length or arc length of the longitudinal strut 14. Preferably, the longitudinal struts 14 are fabricated from nitinol or other shape memory alloy that has a different austenite finish temperature (Af) than that of the circumferential struts 12. Selective application of electricity individually directed at a given longitudinal strut 14 causes the strut 14 to locally exceed austenite finish temperature and the Z will unfold, thus, increasing the longitudinal length of the longitudinal strut 14. The heating is provided by wire elements in the delivery catheter or wire elements within the stent 10, itself. In a preferred embodiment, different wire circuits are provided for each longitudinal strut 14 that might potentially need to be length-adjusted. Thus, the length adjustment is made, post-deployment of the stent 10, from the proximal end of the stent delivery catheter. The wire elements are preferably comprised of a high resistance metal such as, but not limited to, tungsten or nickel-chrome alloy. The electrical couplings to the delivery catheter are either severed when the stent 10 is uncoupled or detached from the delivery catheter, or the electrical couplings remain intact and the electrical heating elements attached to the longitudinal struts 14 by adhesive, magnetic force, or weak mechanical clamping, are pulled free and withdrawn with the delivery catheter.

The heating may also be provided by a secondary catheter that is inserted after delivery of the stent 10 or even after removal of the delivery catheter. The secondary catheter uses ohmic heating or hot water perfused through a balloon to locally heat the longitudinal strut 14 that needs to be lengthened. Preferably, the heating is provided by the delivery catheter so that the stent 10 can be removed if it becomes misplaced. The effects of hysterisis in the heating and cooling response of the nitinol will cause the longitudinal strut 14 to remain in its shape-set length even after the localized heating is removed and the temperature returns to normal body temperature of around 37 degrees centigrade. In yet another embodiment, only pre-determined longitudinal struts 14 are selectively heat treated or configured to expand upon application of heat. Thus, generalized or uniform heating of the entire stent 10 results in only those pre-determined longitudinal struts expanding while the other longitudinal struts 14 do not expand. In yet another embodiment, one or more of the circumferential struts 12 comprises a Z-folded or distorted region that further comprises shape-memory material that has a different Af than that of the rest of the stent 10. In this way, the diameter or effective diameter, of the selected circumferential strut 12 is rendered adjustable.

By way of example, the stent 10 is fabricated from nickel-rich nitinol with an initial Af of 15 degrees Centigrade. The stent 10 is cut to shape. The stent 10 is then placed on a heat-treating mandrel fabricated from P-321 steel. Te heat-treating mandrel maintains the shape of the stent during heat-treating. The stent 10 is heat treated in a sand bath, a salt bath, or an oven, the latter of which preferably including recirculation capabilities. The sand bath or salt bath further comprise a gas injector to bubble inert gas such as, but not limited to, argon, nitrogen, neon, and the like through the sand or salt for the purpose of maintaining even temperature and liquefying the sand or salt. The stent 10 is heat-treated at a temperature of 450 to 550 degrees Centigrade. Preferably, the temperature is held between 500 and 550 degrees Centigrade. The heat-treating time ranges between 1 minute and 15 minutes, preferably ranging between 3 minutes and 10 minutes. Following heat-treating, the stent 10 and the mandrel are submersed in a water bath at approximately room temperature to stop the heat-treating process. By performing this process, the stent 10 has its Af raised from an initial point of 15 degrees Centigrade to the preferred range of 28 to 32 degrees Centigrade. This Af is preferred to allow shape memory expansion of the stent 10 to its full service configuration, following deployment within the body. Process control and process verification are required to empirically determine the exact temperatures and heat-treating times appropriate for the nitinol, taking into account the mass of the mandrel.

At this point, the stent 10 is selectively heat treated to cause certain longitudinal bars 14 to have a higher Af than 28 to 32 degrees Centigrade. Continued application of the heat-treating process causes the Af to increase. This selective heat-treating is performed using a micro-oven into which only the selected longitudinal bars 14 are inserted while the rest of the stent 10 remains outside the micro-oven. The micro-oven may be a simple hot air jet, flame, heated clamp or other device. Preferably, the heat-treating moves the Af of the selected longitudinal bar to a temperature above body temperature, which is typically 36 to 38 degrees Centigrade (mean 37 degrees Centigrade). The preferred temperature range of Af for this embodiment is between 39 and 45 degrees Centigrade. Thus, once the heat is removed, the hysterisis effects of the nitinol will retain the lengthened shape of the selected longitudinal bar 14 even after that bar returns to body temperature. In yet another embodiment, the stent is insulated against temperature in all areas except for the selected longitudinal bar 14 so that, when immersed in a sand bath, salt bath, or oven, only the selected longitudinal bar 14 will remain in the heat-treating temperature range. The insulated bars or struts 12 and 14 will not have their Af appreciably changed during this secondary heat-treating process.

In yet another embodiment, a portion of the stent 10 is coated with a swellable hydrogel material, capable of decreasing the spaces between the individual struts or bars of the sent 10. Referring to FIG. 6C, the hydrogel is preferably selectively coated only at the longitudinally central part of the stent 10 so that the additional obstruction occurs only where the stent 10 is placed across the neck 54 of an aneurysm 51. In yet a further embodiment, the hydrogel is coated onto the stent 10 in a pattern that is not uniform around the circumference of the stent 10. For instance, a plurality of circumferential bars 12 on one side of the stent 10 are coated with hydrogel but the same circumferential bars 12 on the other side of the stent 10, 180 degrees rotated, are not coated with hydrogel. A plurality of the longitudinal bars 14 may also be coated with the hydrogel. The hydrogel is such that the coating will swell from between 1 to 20 times its dry coating thickness on the stent 10 by absorption of water. Preferred hydrogels suitable for this application include those disclosed in U.S. patent application Ser. No. 09/909,715, entitled “Method and Apparatus for Closure of Aneurysm Necks,” the full specification of which is incorporated herein by reference.

FIG. 10A illustrates a distal end view of a catheter connector 100 further comprising an end cap 102, an outer tube 104, a gate 106, a stent 10, and a stent coupler 108. The stent coupler 108 is permanently affixed to the end of the stent 10. The stent coupler 108 is trapped inside the gate 106 and the end cap 102. When the end cap 102 is withdrawn proximally, the stent coupler 108 is free to move laterally through a window in the outer tube 104. The outer tube 104 further comprises a longitudinal slot capable of freely passing the stent coupler 108. The end cap 102 is also slotted permitting proximal end wire of the stent 10 to fall freely outside the constraints of the catheter connector 100.

FIG. 10B illustrates a side cross-sectional view of the catheter connector 100 and a side view, non-sectional, of the proximal end of the stent 10. The catheter connector 100 further comprises an end cap 102, an outer tube 104, a gate 106, a gate pusher 110, a housing cap 112 and a housing pusher 114. The outer tube 104 further comprises a window 118. The stent 10 further comprises a stent coupler 108, a motion stop 116 and a length of wire 120. The motion stop 116 is permanently affixed to the stent 10 and prevents the stent 10 from being withdrawn proximally beyond the window 118 in the outer tube 104 when the gate 106 is withdrawn proximally. The motion stop 116 is configured to stop against the distal end of the end cap 102 at its travel limit. The distance between the proximal end of the motion stop 116 and the distal end of the stent coupler 108 is sufficient to provide a very loose fit around the end cap 102 of the catheter connector 100. The window 118 is sized sufficiently to permit binding free passage of the stent coupler 108. The housing cap 112 is permanently affixed to the proximal end of the outer tube 104 and the housing pusher 114 is an axially elongate cylindrical structure permanently affixed to the housing cap 112. The housing pusher 114 coaxially surrounds the gate pusher 110 and extends the length of the entire stent delivery catheter (not shown) to control mechanisms (not shown) at the proximal end of the delivery catheter. The gate pusher 110 moves axially within the housing pusher 114 and extends to the proximal end of the delivery catheter where it is affixed to control mechanisms (not shown). FIG. 10A shows the gate 106 and the gate pusher 110 in their distally advanced and closed positions. All components of the catheter connector 100 are preferably fabricated from materials such as, but not limited to, stainless steel, cobalt-nickel alloys, nitinol, Elgiloy, MP-35N, and the like. This type of catheter connector 100 is suitable for a stent 10 wherein the primary structure is a length of wire 120 that is deformable for delivery and expands to take on its pre-determined shape following delivery. The catheter connector 100 is configured to controllably hold the stent 10 until such time as it is desired to release or disconnect the stent 10.

FIG. 10C illustrates a side cross-sectional view of the catheter connector 100 and a side view, non-sectional, of the proximal end of the stent 10. The catheter connector 100 further comprises an end cap 102, an outer tube 104, a gate 106, a gate pusher 110, a housing cap 112, and a housing pusher 114. The outer tube 104 further comprises a window 118. The stent 10 further comprises a stent coupler 108, a motion stop 116, and a length of wire 120. In FIG. 10C, the gate 106 and the gate pusher 110 are shown withdrawn proximally to their open configurations, relative to the outer tube 104 and the housing pusher 114, such that the stent coupler 108 is free to move laterally past the window 118 in the outer tube 104 and thus be released or detached.

FIG. 11A illustrates the distal tip of a stent delivery catheter 31 further comprising a stent 10, a sheath 130, a plurality of catheter connectors 100, central pusher 132, and a pusher hook 134. The stent 10 further comprises a length of stent wire 120. The stent delivery catheter 31 is configured to deliver a stretchable, or elongatable, wire stent 10 to a terminal bifurcation such as a basilar tip aneurysm via the vertebral arteries. The stent wire 120 is folded on itself and held at its center by the pusher hook 134, which is further affixed to the central pusher 132. Referring to FIGS. 11A, 10B, and 10C, the two ends of the wire 120 are releasably affixed to the catheter connectors 100. The stent 10 is advanced out the distal tip of the sheath 130 and its center is located at or inside the aneurysm. The catheter connectors 100 continue to advance as the sheath 130 is retracted causing the two ends of the stent wire 120 to emerge from the sheath 130 and form their axially elongate cylindrical stent 10 shape to obstruct the inflow to the aneurysm. Again referring to FIGS. 11A, 10B, and 10C, the pushers attached to the catheter connectors 100 are pre-bent to form a “J” shape once they emerge from the sheath 130 to assist in coercing the stent wire 120 ends into the branch vessels of the bifurcation.

FIG. 11B illustrates an end view of the distal tip of the stent delivery catheter 31 further comprising a sheath 130, a plurality of stent connectors 100, a plurality of ends of stent wire 120, and a central pusher 132. The central pusher 132 is partially deployed and is shown in cross-section as it extends beyond the distal end of the sheath 130. The stent wires 120 are also shown in cross-section as they extend beyond the distal tip of the sheath 130.

FIG. 11C illustrates a side view of the proximal end of a stent delivery catheter 31, further comprising a microcatheter attachment 160, a stabilization arm 142, a sheath 130, a stent pusher 150, a gate pusher 158, a linear gear 144, a first gear 146, a second gear 148, a housing 156, a pusher reel 154, a sheath traveler 162, a linear bushing 164, and a link belt 152. The microcatheter attachment 160 is removable affixed to the proximal hub of a microcatheter 140.

Referring to FIG. 11C, the housing 156 provides the reference against which all components are mounted. The pusher reel 154 is rotatably affixed to the housing 156 by a reel bearing. The stent pusher 150 and internally coaxial gate pusher 158 are wound onto the pusher reel 154. The pusher reel 154 is attached to the link belt 152 that is further attached to the second gear 148. The second gear 148 is rotatably affixed to the housing 156 and engages with the first gear 146, which is also rotatably affixed to the housing 156. The first gear 146 and the second gear 148 are affixed to the housing 156 by rotational bearings. The first gear 146 also engages with a linear gear 144, which is permanently affixed to the sheath traveler 162. The sheath traveler 162 slides within the housing 156 with a travel distance at least as long as that of the deployed stent. The sheath traveler 162 slides smoothly within the housing 156 on the linear bushing 164, which is affixed to the housing 156. The stabilization arm 142 is permanently affixed to the microcatheter attachment 160, which is removable attached to the microcatheter 140 by a luer lock, bayonet mount, screw thread or other reversible locking mechanism. Many components of the proximal end of the delivery catheter 31 are preferably fabricated from polymeric materials such as, but not limited to, ABS, PVC, polycarbonate, polysulfone, polyamide, polyacetal, polyolefin, and the like. Metallic components, typically but not necessarily fabricated from stainless steel or cobalt nickel alloys, are also acceptable in this embodiment.

The stent delivery catheter 31 is configured as shown in FIG. 11C to permit controlled delivery of the stretchable stent 10 into the vasculature. The stent 10 is much longer in the catheter 31 than it is following deployment. For example, the stent of FIG. 1A, with an outer deployed diameter of 4 mm, a deployed length of 40 mm and circumferential elements 12 spaced 4 mm apart would require approximately 178 mm of wire. The sheath 130 is withdrawn at a rate approximately ⅓ that which the catheter connector 100 is advanced so that for every 4 mm of deployed stent 10 configuration that is exposed, approximately 16 mm of wire is deployed out the distal end of the sheath 130. The system is further configured to stabilize the distal end of the deployed stent 10 relative to the microcatheter 140, and thus the anatomy. All motion is kept relative to this point of reference so that control is transparent to the user. Either the first gear 146 or the second gear 148 are the primary drive for the system. The primary drive gear of these interconnected gears are driven either by electric motor with a user control (linear or proportional), by a ratchet lever, by a knob, by a reel, by a trigger with ratchet, or other mechanism. Preferably, the trigger with ratchet drive is used to permit one-handed operation of the system. The sheath traveler 162 slides within the housing 156 on the linear bearing or bushing 164. A bushing 164 is preferably fabricated from a low friction material such as Teflon or FEP. FEP is preferred over Teflon (polytetrafluoroethylene) because it is capable of being radiation sterilized at effective levels without degrading. Smooth metal surfaces are also suitable for this application. The linear bushing 164 may also be fabricated as a bearing using roller bearings, ball bearings or the like.

The present invention may be embodied in other specific forms without departing from its spirit or essential characteristics. For example, the stents may be self-expanding or they may be balloon expandable. The stents may be completely or partially bioresorbable with the bioresorbable components fabricated from materials such as, but not limited to, polylactic acid or polyglycolic acid. The stents may be used for cerebrovascular aneurysms or major vessel aneurysms or dissections. Many specific details may vary while maintaining the essence of the invention. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. An implantable device for bridging the neck of an aneurysm, comprising:
a plurality of circumferential bars, and
a plurality of longitudinal bars,
wherein the longitudinal bars further comprise notches or bends to improve flexibility.

2. The stent of claim 1 wherein the circumferential bars are disposed orthogonally to the longitudinal axis of the stent.

3. The stent of claim 1 wherein the circumferential bars are disposed in a spiral pattern relative to the longitudinal axis of the stent.

4. The stent of claim 1 wherein the circumferential bars are disposed at an angle other than 90 degrees relative to the longitudinal axis of the stent.

5. The stent of claim 1 wherein the distance between circumferential bars is greater at the ends of the stent than toward the center of the stent.

6. The stent of claim 1 wherein the width of the circumferential bars is greater toward the center of the stent than at the ends of the stent.

7. The stent of claim 1 wherein the width of the longitudinal bars is greater toward the center of the stent than at the ends of the stent.

8. The stent of claim 1 wherein at least a portion of the circumferential bars are coated with a swellable hydrogel.

9. The stent of claim 1 wherein at least a portion of the longitudinal bars are coated with a swellable hydrogel.

10. The stent of claim 1 wherein the axially central circumferential or longitudinal bars are coated with a swellable hydrogel.

11. The stent of claim 1 wherein one or more of the longitudinal bars is Z-folded or bent.

12. The stent of claim 11 wherein the Z-folded or bent longitudinal bars are fabricated from shape-memory alloy.

13. The stent of claim 11 wherein the Z-folded or bent longitudinal bars are fabricated from nitinol.

14. The stent of claim 11 wherein the Z-folded or bent longitudinal bars are selectively heated to cause unfolding and result in an increase in longitudinal dimension.

15. A stent, adapted for bridging the neck of an aneurysm, comprising:
an inner helix, and
an outer helix,
wherein the inner and outer helix are separate structures.

16. The stent of claim 15 wherein the inner helix and the outer helix further comprise a plurality of filaments.

17. The stent of claim 15 further comprising a coating to improve radiopacity.

18. The stent of claim 17 wherein the coating is fabricated, at least in part, from tantalum, platinum or gold.

19. The stent of claim 15 wherein the inner helix and the outer helix are counterwound.

20. The stent of claim 15 further comprising a delivery catheter that controllably counterwinds the inner helix and outer helix relative to each other to collapse the stent for delivery to the patient.

21. The stent of claim 15 further comprising a delivery catheter that controllably counterwinds the inner helix and the outer helix relative to each other and stretches the inner helix and the outer helix to compress longitudinally within the delivery catheter.

22. The stent of claim 1 further comprising a delivery catheter that winds the stent to a diameter smaller than its expanded diameter.

23. The stent of claim 1 further comprising a delivery catheter that selectively locks and unlocks from the stent.

24. The stent of claim 1 further comprising a delivery catheter that stretches the stent into a generally longitudinal configuration for delivery in the smallest possible profile and with the greatest possible flexibility.

25. A method of treating cerebrovascular aneurysms comprising the steps of:
accessing the aneurysm with a guide catheter and a guidewire,
delivering a stent to cover the neck of the aneurysm but minimizing coverage of feeder vessels and branch vessels,
delivering embolic material through the openings in the stent to fill and pack the aneurysm, and
adjusting the length of one or more longitudinal bars in the stent through selective heating of the bars.

26. The method of claim 25 further comprising the step of inserting a secondary catheter to selectively heat the longitudinal bars.

27. The method of claim 25 wherein the step of adjusting the length of the longitudinal bars is performed under fluoroscopic guidance.

28. The method of claim 25 wherein the longitudinal bar lengths are adjusted with a catheter that heats the entire stent uniformly.

29. The method of claim 28 further comprising the precursor step of heat-treating only pre-determined bars so that they lengthen upon application of heat.

30. The stent of claim 1 further comprising a delivery catheter further comprising a coupler or attachment to the stent that may be selectively uncoupled from the proximal end of the delivery catheter.

31. The stent of claim 30 wherein the coupler holds the stent with a reversible mechanical interference.

32. The stent of claim 30 wherein the coupler holds the stent with a fusible link.

33. The stent of claim 30 wherein the coupler holds the stent with a shape-memory actuator operated mechanical interference.

34. The stent of claim 30 wherein the coupler holds the stent with a friction joint that is overcome by application of hydraulic pressure.
